(19) United States
(12) Patent Application Publication
(10) Pub. No.: US 2015/0374865 A1
(43) Pub. Date: Dec. 31, 2015

(54) ATMOSPHERIC MICROWAVE STERILIZERS AND METHODS

(71) Applicant: Xanatize LLC, Park Ridge, NJ (US)

(72) Inventor: Edward R. Peterson, Pearland, TX (US)

(21) Appl. No.: 14/747,256

(22) Filed: Jun. 23, 2015

Related U.S. Application Data
(60) Provisional application No. 62/016,941, filed on Jun. 25, 2014.

(51) Int. Cl. A61L 2/18

(52) U.S. Cl. CPC A61L 2/18

(57) ABSTRACT

An apparatus for sterilizing instruments at atmospheric pressure is provided. The apparatus includes a receiver, a sterilant reservoir, a rinsant reservoir, a waste reservoir, a microwave source, and a controller. The receiver receives the instruments to be sterilized and is made of a non-microwave absorptive material. The sterilant reservoir contains a sterilant fluid that is absorptive of microwave energy and has a boiling point greater than 100° Celsius. The rinsant reservoir contains a chemical rinsant fluid that is capable of solvating or displacing the sterilant fluid and does not support microbial viability or growth. The microwave source is positioned with respect to receiver so as to emit microwave energy into the receiver. The reservoirs are each in selective fluid communication with the receiver and the controller selectively moves the sterilant fluid or the chemical rinsant fluid from the reservoirs to the receiver and from the receiver to the waste reservoir.

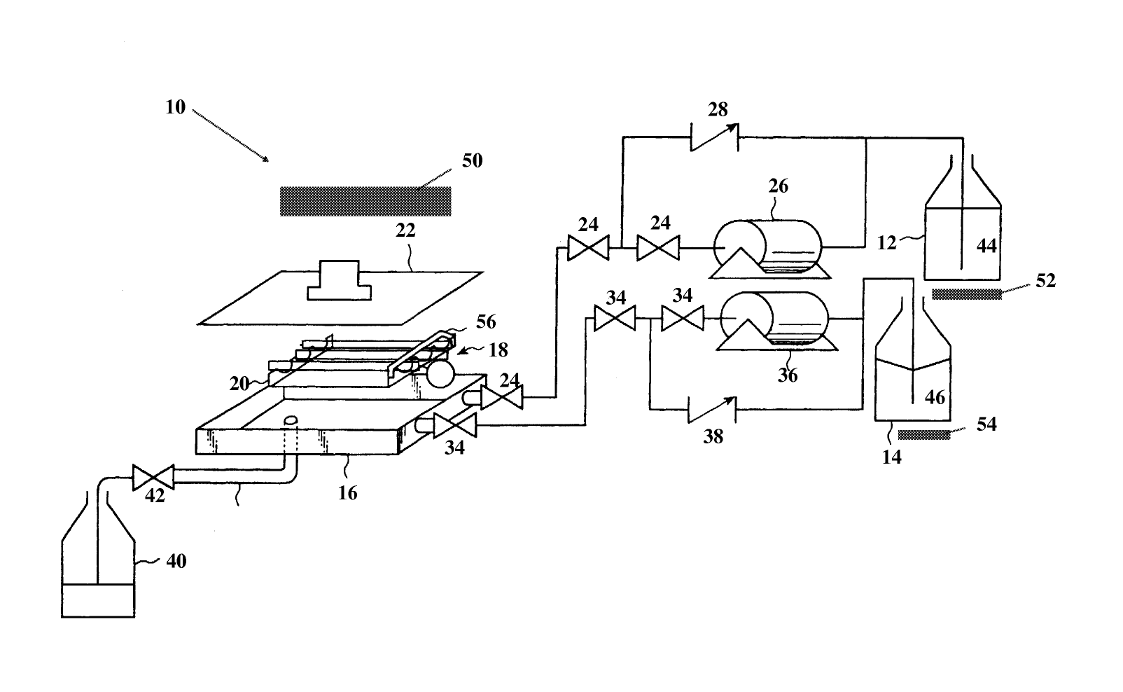

### CROSS REFERENCE TO RELATED APPLICATIONS

This application claims the benefit of U.S. Provisional Application Ser. No. 62/016,941 filed Jun. 25, 2014, the entire contents of which are incorporated by reference herein.

### BACKGROUND

1. Field of the Invention

The present disclosure relates to a microwave sterilizers and microwave sterilizing methods, which are configured to sterilize at atmospheric pressure instruments or devices such as but not limited to medical instruments.

2. Description of Related Art

Sterilization is defined as the complete destruction of microorganisms and is generally accomplished by exposure to one or more of heat, chemical compounds, radiation, and any combination thereof.

Each of these prior art methods has its benefits and its drawbacks. For example, autoclaves have been used for many years to sterilize medical devices. Such autoclaves have a pressure vessel in which the device or product to be sterilized is placed. The air within the chamber is replaced with steam at a desired temperature and pressure. Typical autoclaves operate at a temperature of 121° C. or more and pressurized to 15 pounds per square inch. Of course other types of autoclaves, commonly known as overpressure or counter pressure autoclaves, operate at much higher temperatures and/or pressures. The device is maintained at the desired sterilization parameters for a predetermined period of time. Unfortunately, the high pressures associated with the operation of the prior art autoclaves can increase the cost to own, operate, and maintain such autoclaves.

More recently, microwave sterilizers have been developed that operate at ambient pressures. One such microwave sterilizer is described in U.S. Pat. No. 5,759,486, the contents of which are incorporated by reference in their entirety herein. Here, medical instruments are sterilized at atmospheric pressure using microwave energy to heat liquids with high boiling points to effect sterilization. Unfortunately, such prior art microwave sterilizers require a rinsing step in which the high boiling liquid is rinsed away using sterilized water that has proven to reduce the utility of such sterilizers.

### SUMMARY

An apparatus for sterilizing instruments at atmospheric pressure is provided. The apparatus includes a receiver, a sterilant reservoir, a rinsant reservoir, a waste reservoir, a microwave source, and a controller. The receiver receives the instruments to be sterilized and is made of a non-microwave absorptive material. The sterilant reservoir contains a sterilant fluid that is absorptive of microwave energy and has a boiling point greater than 100° Celsius. The rinsant reservoir contains a chemical rinsant fluid that is capable of solvating or displacing the sterilant fluid and does not support microbial viability or growth. The microwave source is positioned with respect to receiver so as to emit microwave energy into the receiver. The reservoirs are each in selective fluid communication with the receiver and the controller selectively moves the sterilant fluid or the chemical rinsant fluid from the reservoirs to the receiver and from the receiver to the waste reservoir.

In some embodiments alone or in combination with one or more of the aft mentioned embodiments, the controller activates the microwave source when the instruments in the receiver are immersed in the sterilant fluid to heat and maintain the sterilant fluid via the microwave energy at a desired temperature for a desired period of time sufficient to sterilize the instruments.

In other embodiments alone or in combination with one or more of the afore and/or aft mentioned embodiments, the controller rinses the instruments in the receiver with the chemical rinsant fluid after the instruments have been sterilized.

In other embodiments alone or in combination with one or more of the afore and/or aft mentioned embodiments, the apparatus further includes a cover removably secured to the receiver to cover the instruments received therein at atmospheric pressure.

In other embodiments alone or in combination with one or more of the afore and/or aft mentioned embodiments, the cover is made of a microwave transparent material so that the microwave energy from the microwave source heats the sterilant fluid.

In other embodiments alone or in combination with one or more of the afore and/or aft mentioned embodiments, the microwave transparent material is selected from the group consisting of glass, plastic, and ceramic.

In other embodiments alone or in combination with one or more of the afore and/or aft mentioned embodiments, the apparatus further includes a tray received in the receiver, where the tray holds the instruments.

In other embodiments alone or in combination with one or more of the afore and/or aft mentioned embodiments, the apparatus further includes a metal strip connecting the tray to the instruments.

In other embodiments alone or in combination with one or more of the afore and/or aft mentioned embodiments, the chemical rinsant fluid is selected from the group consisting of methanol, ethanol, propanol, trichlorofluoromethane (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a), ethyl chloride (R-160), methyl formate (R-611), acetone, diethyl ether, ethyl ether, methyl ethyl ether, and any combinations thereof.

In other embodiments alone or in combination with one or more of the afore and/or aft mentioned embodiments, the apparatus further includes a temperature control device in a heat exchange relationship with the sterilant reservoir, which preheats the sterilant fluid under the control of the controller before movement to the receiver.

In other embodiments alone or in combination with one or more of the afore and/or aft mentioned embodiments, the apparatus includes a temperature control device in a heat exchange relationship with the rinsant reservoir, which cools the chemical rinsant liquid under the control of the controller before rising the instruments.

An atmospheric pressure, microwave sterilization process is also provided. The method includes placing instruments in receiver; immersing the instruments in a sterilant fluid within the receiver, the sterilant fluid being absorptive of microwave energy and having a boiling point greater than 100° C.; directing microwave energy into the sterilant fluid for a sufficient time to increase and the sterilant fluid to a sterilization temperature; draining the sterilant fluid from the receiver after; and rinsing the instruments with a chemical rinsant liquid that is capable of solvating or displacing the sterilant fluid and does not support microbial viability or growth.

In other embodiments alone or in combination with one or more of the afore and/or aft mentioned embodiments, the chemical rinsant fluid is selected from the group consisting of methanol, ethanol, propanol, trichlorofluoromethane (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a), ethyl chloride (R-160), methyl formate (R-611), acetone, diethyl ether, ethyl ether, methyl ethyl ether, and any combinations thereof.

In other embodiments alone or in combination with one or more of the afore and/or aft mentioned embodiments, the method further includes preheating the sterilant fluid before immersing the instruments in the sterilant fluid within the receiver.

In other embodiments alone or in combination with one or more of the afore and/or aft mentioned embodiments, the method further includes cooling the chemical rinsant liquid before rinsing the instruments so that the chemical rinsant liquid cools the instruments from the sterilization temperature.

The above-described and other features and advantages of the present disclosure will be appreciated and understood by those skilled in the art from the following detailed description, drawings, and appended claims.

### DESCRIPTION OF DRAWING

The FIGURE illustrates an exemplary embodiment of a microwave sterilizer according to the present disclosure.

### DETAILED DESCRIPTION

Referring to the sole Figure, an exemplary embodiment of a microwave sterilizer according to the present disclosure is shown and is referred to by reference numeral 10.

Generally, sterilizer 10 is configured to immerse instruments in a sterilant fluid, heat the sterilant fluid via microwave energy to a sufficient temperature and for a sufficient time so as to sterilize the instruments at atmospheric pressure, and then to rinse the sterilant from the instruments using a chemical rinsant. It has advantageously been determined by the present disclosure that the use of a chemical rinsant, instead of water, eliminates the need to sterilize the water and to maintain the water in a sterile state.

Sterilizer 10 includes a first reservoir 12 and a second reservoir 14 each in selective fluid communication with a receiver 16. Receiver 16 is sized and configured to receive one or more instruments 18 such as, but not limited to, medical, surgical, dental, veterinary or other instruments. In some embodiments, instruments 18 can be placed on a tray 20, which is then received in receiver 16. Once instruments 18 are received in receiver 16, with or without tray 20, receiver 16 can be closed by a door or cover 22. Optionally, a gasket (not shown) may be inserted or placed between door 22 and receiver 16 in order to form a better liquid seal.

First reservoir 12 is in selective fluid communication with receiver 16 for example by way of one or more valves 24, a pump 26, and a fluid flow check valve 28. Similarly, second reservoir 14 is in selective fluid communication with receiver 16 for example by way of one or more valves 34, a pump 36, and a fluid flow check valve 38. Finally, receiver 16 is in selective fluid communication with a waste reservoir 40 via one or more valves 42.

First reservoir 12 includes a supply of a sterilant fluid 44. Sterilant fluid 44 is absorptive of microwave energy and has a boiling point greater than 100° Celsius, preferably at least 121° Celsius, and more preferably at least 140° Celsius. Sterilant fluid 44 may contain water due to addition, contamination, or absorption from air, but will be less than 50% water. Additionally, sterilant fluid 44 has a higher electrical resistance, higher thermal resistance, and lower volatility when compared to water or weak solutions comprised mostly of water. In addition, desirable properties of the sterilant fluid 44 include higher viscosity and lower heat capacity than water.

Examples of sterilant fluids 44 contemplated for use by the present disclosure include, but are not limited to, Polyethylene glycol, Propylene glycol, Glycerin, Di(propylene glycol), 2,2-Dimethyl-1-3,butanediol, Triethylene glycol, Tetraethylene glycol, Dimethyl sulfoxide, Triethanolamine, Triethylcitrate, Tetrahydrofurfuryl acetate, Thiodiglycol, Propyleneglycol phenyl ether, 1-Heptanol, Methane Sulfonic acid, Diethylene triamine, N,N-Dimethylformamide Glutaraldehyde, Propiolactone, Diiodomethane, aniline (363F), Dowtherm (496F), Bromobenzene (313F), ethylene bromide (269F), nitrobenzene(412F), hexachloroethane, compounds containing chlorine, compounds containing fluorine, Hexachloroethane (365 F, R-110), pentacloroethane (324 F, R-120) and pentachloromonofluoroethane (279 F, R-111), and any combinations thereof.

Many chemicals are not absorptive of microwave energy in their pure state because they have a symmetrical structure, such as octane, hexachlorobenzene and carbon tetrachloride. However, adding even small amounts of microwave absorbing chemicals to a microwave non-absorptive chemical makes the entire homogeneous material microwave absorptive. Therefore, hexachloroethane which is symmetrical and not microwave absorptive can be made microwave absorptive if a small amount (1% to 5% or more) of pentachloroethane or similar material is added to it. Thereby, microwave absorptive chemicals need not be the primary component of a mixture that is used as the sterilant fluid. For example, high temperature chemicals containing chlorine and fluorine could be used as a sterilant. Examples are (with boiling point, refrigerant type): Hexachloroethane (365 F, R-110), pentacloroethane (324 F, R-120) and pentachloromonofluoroethane (279 F, R-111) which are commercial refrigerants. These would be paired with rinsants that are similar in chemical nature but of much lower boiling point such as (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a).

Additionally, food grade (or better) oils, resins, waxes, flavorants, gums, essential oils, or other plant and animal products that have a boiling temperature above 100° Celsius are meant to be included as sterilant fluid 44.

Second reservoir 14 includes a supply of a rinsant fluid 46. Rinsant fluid 46 can be any non-biogenic fluid or antiseptic that is capable of killing microbes, as well as being able to effectively rinse away the sterilant fluid 44. Rinsant 46, preferably, has a relatively low toxicity and are generally known to be safe for human contact.

Examples of rinsant fluids 46 contemplated for use by the present disclosure include, but are not limited to, methanol, ethanol, propanol, trichlorofluoromethane (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a), ethyl chloride (R-160), methyl formate (R-611), acetone, diethyl ether, ethyl ether, methyl ethyl ether, and any combinations thereof. The rinsant fluid 46 may be a mixture containing a relatively low boiling compound with another, with a third chemical or with water, such that the mixture is effective as a rinsant and is non-biogenic or antiseptic. Rinsants may also comprise known or suspected anti-septic compounds, solutions or mixtures for additional effectiveness.

As used here, the term “rinsant” is defined as a liquid chemical material that can remove the sterilant fluid and not introduce or re-introduce microbial life directly to the medical instruments or allow microbes to remain viable. To be considered a rinsant according to the present disclosure, the chemicals must both be capable of solvating or displacing the sterilant fluid subsequent to the sterilization step as well as not support microbial viability or growth while the rinsant is used or thereafter during cooling the instruments to use temperature while the instrument is encapsulated by the rinsant. A rinsant will be chosen by its ability to remove the sterilizing chemical chosen. Some rinsants will have mild anti-biotic or sterilizing character while others will simply be non-supportive of biological growth. All rinsants will be introduced to the sterilizing chamber such that the chamber and instruments are cooled.

In preparation for sterilization, valves 24, 42, pump 26, fluid flow check valve 28 are controlled—by for example a controller—to transfer a predetermined amount of sterilant fluid 44 from first reservoir 12 into receiver 16 until the instruments are submerged in the sterilant fluid. Once submerged, the instruments are in intimate thermal contact with the sterilant fluid, assuring that the entire surface of the instrument is exposed to the same temperature during the sterilization process as well as the same chemical environment.

Sterilizer 10 further includes a microwave source 50 configured to heat and maintain sterilant fluid 44 to/at a desired temperature. Thus, sterilizer 10 activates microwave source 50 for a long enough period of time to allow the sterilant fluid 44 to reach and maintain a specific temperature for sterilization. Once the specific temperature has been attained, the temperature is maintained for a specific period of time. The entire process for the heating of the sterilant fluid 44 and maintenance of the temperature is referred to herein as the sterilization heating process.

At the conclusion of the sterilization heating process, i.e., when the temperature has been maintained for the specific period of time, sterilizer 10 deactivates microwave source 50 and opens valve 42 to allow sterilant fluid 44 to drain from receiver 16 into third reservoir 40. Of course, it is contemplated by the present disclosure for sterilizer 10 to include a pump or other fluid removal system (not shown) sufficient to remove sterilant fluid 44 from receiver 16.

Once sterilant fluid 44 has drained or otherwise been removed from receiver 16, valves 34, 42, pump 36, and fluid flow check valve 38 are controlled—by for example the controller—to transfer a predetermined amount of rinsant fluid 46 from second reservoir 14 into receiver 16 in such a manner that the rinsant fluid 46 rinses sterilant fluid 44 from instruments 18 as well as cools them. As necessary, sterilizer 10 opens valve 42 to allow the rinsant fluid 46 to drain from receiver 16 into third reservoir 40. The entire process for the rinsing the sterilant fluid 44 from instruments 18 and any cooling of the instruments and other components of sterilizer 10 is referred to herein as the rinsing process.

Importantly, sterilizer 10 remains at ambient pressure during the filling/draining of sterilant fluid 44, filling/draining of rinsant fluid 46, heating and maintaining of the temperature of the sterilant fluid 44, and the rinsing/cooling with the rinsant fluid 46.

For example, sterilizer 10 can include a pressure relief system (not shown) in receiver 16 and/or in door 22, or in any other desired location, to allow any expansion or contraction of the atmosphere within receiver 16 (when closed by door 22) caused by the filling, heating and rinsing to vent from or into the receiver.

In some embodiments, the pressure relief system can simply be a two way pressure relief valve. In some embodiments, the pressure relief system can—at least when drawing air into the receiver 16—can be a sterile vent that filters any ambient air as it is drawn into the sterilizer.

It is contemplated by the present disclosure for the pressure relief system to be a more complex system such as a pressure sensor, a pressurized source of inert gas source, and a vacuum pump, which work to add or withdraw atmosphere from receiver 16 as needed to maintain sterilizer 10 at ambient pressure.

As used herein, the term “ambient pressure” shall mean any pressure gradient between the internal and external areas of receiver 16 so as to ensure that sterilizer 10 not considered to be a pressure vessel as that term is understood and defined by those skilled in the art as evidenced by, for example, the International Boiler and Pressure Vessel Code 2013.

In some embodiments, sterilizer 10 can include can include a temperature control device in heat exchange relationship with first and/or second reservoirs 12, 14.

For example, sterilizer 10 can include a temperature control device 52 in a heat exchange relationship with first reservoir 12 and in electrical communication with the controller—where the controller controls the temperature control device to maintain sterilant fluid 44 at a predetermined storage temperature before use and/or configured to preheat the sterilant fluid 44 before transfer to receiver 16, which can decrease the time necessary to complete the sterilization heating process.

Additionally or alternatively, sterilizer 10 can include a temperature control device 54 in a heat exchange relationship with second reservoir 14 and in electrical communication with the controller—where the controller controls the temperature control device to maintain rinsant fluid 46 at a predetermined storage temperature before use and/or configured to cool the rinsant fluid 46 before transfer to receiver 16, which can decrease the time necessary to complete the cooling from the rinsing process.

After instruments 18 have been sterilized, rinsed and, when desired, cooled, receiver 16 can be opened by removing or otherwise uninstalling or opening cover 22 so that tray 20 holding the instruments 18 can be removed from the receiver 16.

In some embodiments, the tray 20 and the receiver 16 are made from stainless steel (a non-microwave absorptive metal) and the cover 22 is made from borosilicate glass (a microwave transparent material) to allow microwave energy from microwave source 50 to penetrate and heat sterilant fluid 44. The metal tray 20 and receiver 16 allow for rapid cool-down, reduction or elimination of the potential for arcing, and the use of fluids that reach temperatures from greater than 100° Celsius to greater than 300° Celsius before boiling. A metal strip 56 can be used to connect metal tray 20 to instruments 18, when the instruments are made of metal, thereby maintaining the same potential and removing the possibility for arcing in sterilizer 10.

Of course, it is contemplated by the present disclosure for tray 20 and/or receiver 16 to be made from glass, plastic, ceramic or any other microwave transparent material that allows microwaves to penetrate into and heat the sterilant fluid 44 contained in receiver 16. Examples of such materials include polyetherimides, polyimides, Kalrez and similar polymers, polytetrafluoroethylene, quartz, Pyrex glass, dense aluminum oxide, etc.

If the receiver 16 is made from glass, plastic, ceramic or any other microwave transparent material, the cover 22 can be made from any non-microwave absorptive metal or microwave transparent material. In no case can the entire container consisting of the receiver 16 and the cover 22 be made from metal that does not absorb microwave energy without a means for introducing microwave energy into sterilant fluid 44 contained therein.

The present embodiment of the high temperature, non-conductive fluid, super heating fluid microwave sterilizer passes the sterilant fluid 44 and/or rinsant fluid 46 through the sterilizer 10 once. However, it is contemplated by the present disclosure for sterilizer 10 to include one or more recapture systems (not shown) to recapture only sterilant fluid 44 and feed the recaptured sterilant fluid into first reservoir 12, to recapture only rinsant fluid 46 and feed the recaptured rinsant fluid into second reservoir 14, to recapture sterilant and rinsant fluids together as is shown in the Figure, or to recapture both sterilant and rinsant fluids separately and feed the recaptured fluid to the appropriate reservoir 12, 14.

In other embodiments where sterilizer 10 utilizes a rinsant fluid 46 that has some toxicity, the sterilizer 10 can include a vacuum vapor removal system attached to or surrounding the device such that all or nearly all fumes are removed from the sterilized medical tools and the fumes are removed from working or use areas.

The high temperature, non-conductive fluid, super heating fluid microwave sterilizer and the atmospheric pressure, high temperature microwave sterilization process of the present disclosure overcome the problems associated with conventional autoclave and microwave sterilization apparatuses and procedures. The fluids used in the present disclosure do not boil at the temperatures used and there is no need to contain pressure in the sterilization process. The microwave oven does not get hot and is not itself dangerous. Only the fluid and the fluid container with the tools and/or instruments inside get hot. This smaller heated mass means a shorter warm-up time and a shorter cool-down time. Moreover, the cool-down time is further reduced by rinsing the tools and/or instruments with rinsant fluid, which can remain on the instruments after rising.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, the present disclosure is not intended to be limited to the particular embodiment(s) disclosed as the best mode contemplated.

What is claimed is:

1. An apparatus for sterilizing instruments at atmospheric pressure, comprising:
a receiver configured to receive the instruments to be sterilized, the receiver being made of a non-microwave absorptive material;
a sterilant reservoir containing a sterilant fluid that is absorptive of microwave energy and has a boiling point greater than 100° Celsius, the sterilant reservoir being in selective fluid communication with the receiver;
a rinsant reservoir containing a chemical rinsant fluid that is capable of solvating or displacing the sterilant fluid and does not support microbial viability or growth, the rinsant reservoir being in selective fluid communication with the receiver;
a waste reservoir being in selective fluid communication with the receiver;
a microwave source positioned with respect to receiver so as to emit microwave energy into the receiver; and
a controller configured to selectively move a desired amount of the sterilant fluid from the sterilant reservoir to the receiver, to selectively move the sterilant fluid from the receiver to the waste reservoir, to selectively move the chemical rinsant fluid from the rinsant reservoir to the receiver, and to selectively move the chemical rinsant fluid from the receiver to the waste reservoir,
wherein the controller is further configured to activate the microwave source when the instruments in the receiver are immersed in the sterilant fluid to heat and maintain the sterilant fluid via the microwave energy at a desired temperature for a desired period of time sufficient to sterilize the instruments, and
wherein the controller is further configured to rinse the instruments in the receiver with the chemical rinsant fluid after the instruments have been sterilized.

2. The apparatus of claim 1, further comprising a cover removably secured to the receiver to cover the instruments received therein at atmospheric pressure.

3. The apparatus of claim 1, wherein the cover is made of a microwave transparent material so that the microwave energy from the microwave source heats the sterilant fluid

4. The apparatus of claim 3, wherein the microwave transparent material is selected from the group consisting of glass, plastic, and ceramic.

5. The apparatus of claim 1, further comprising a tray received in the receiver, the tray being configured to hold the instruments.

6. The apparatus of claim 5, further comprising a metal strip connecting the tray to the instruments.

7. The apparatus of claim 1, wherein the chemical rinsant fluid is selected from the group consisting of methanol, ethanol, propanol, trichlorofluoromethane (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a), ethyl chloride (R-160), methyl formate (R-611), acetone, diethyl ether, ethyl ether, methyl ethyl ether, and any combinations thereof.

8. The apparatus of claim 1, further comprising a temperature control device in a heat exchange relationship with the sterilant reservoir, the controller controlling the temperature control device to preheat the sterilant fluid before movement to the receiver.

9. The apparatus of claim 1, further comprising a temperature control device in a heat exchange relationship with the rinsant reservoir, the controller controlling the temperature control device to cool the chemical rinsant liquid to a desired temperature before movement to the receiver to cool the instruments during rinsing.

10. An atmospheric pressure, microwave sterilization process, comprising:
placing instruments in receiver;
immersing the instruments in a sterilant fluid within the receiver, the sterilant fluid being absorptive of microwave energy and having a boiling point greater than 100° C.;
directing microwave energy into the sterilant fluid for a sufficient time to increase and the sterilant fluid to a sterilization temperature;
draining the sterilant fluid from the receiver after; and
rinsing the instruments with a chemical rinsant liquid that is capable of solvating or displacing the sterilant fluid and does not support microbial viability or growth.

11. The method of claim 10, wherein the chemical rinsant fluid is selected from the group consisting of methanol, ethanol, propanol, trichlorofluoromethane (R-11), dichlorofluoromethane (R-21), monochlorofluoromethane (R-31), chloromethane (R-40), monochlorotetrafluoroethane (R-124), monochlorotrifluoroethane (R-133a), tetrafluoroethane (R-134a), ethyl chloride (R-160), methyl formate (R-611), acetone, diethyl ether, ethyl ether, methyl ethyl ether, and any combinations thereof.

12. The method of claim 10, further comprising preheating the sterilant fluid before immersing the instruments in the sterilant fluid within the receiver.

13. The method of claim 10, further comprising cooling the chemical rinsant liquid before rinsing the instruments so that the chemical rinsant liquid cools the instruments from the sterilization temperature.
